(19) United States
(12) Patent Application Publication
(10) Pub. No.: US 2025/0213786 A1
(43) Pub. Date: Jul. 3, 2025

(54) WIRELESS CONTROL OF A DRUG DELIVERY DEVICE

(71) Applicant: Tandem Diabetes Care, Inc., San Diego, CA (US)

(72) Inventor: Michael Rosinko, Anaheim, CA (US)

(21) Appl. No.: 19/086,378

(22) Filed: Mar. 21, 2025

Related U.S. Application Data
(63) Continuation of application No. 17/023,995, filed on Sep. 17, 2020.
Application No. 17/023,995 is a continuation of application No. 16/273,850, filed on Feb. 12, 2019, now Pat. No. 10,806,851.
Application No. 16/273,850 is a continuation of application No. 15/336,930, filed on Oct. 28, 2016, now Pat. No. 10,213,547.
Application No. 15/336,930 is a continuation of application No. 14/581,461, filed on Dec. 23, 2014, now Pat. No. 9,486,571.
(60) Provisional application No. 61/920,940, filed on Dec. 26, 2013.

(51) Int. Cl. A61M 5/142; G08C 17/02; G16H 20/17; G16H 40/40; G16H 40/67

(52) U.S. Cl. CPC A61M 5/14244; G08C 17/02; G16H 20/17; G16H 40/40; G16H 40/67; A61M 2205/12; A61M 2205/3576; A61M 2205/3584; A61M 2205/3592; A61M 2205/50; A61M 2205/502; A61M 2205/52; A61M 2230/201; G08C 2201/93

(57) ABSTRACT

A system and method provide for enhanced reliability and safety of programming and/or operating a medical device, such as an infusion pump, with a remote control device, such as a mobile phone (e.g., a smartphone). A processor can determine whether the smartphone is compatible with the medical device by checking the type and version of the smartphone as well as the versions of the operating software and/or firmware resident on the phone.

### RELATED APPLICATIONS

This application is a continuation of application Ser. No. 17/023,995 filed Sep. 7, 2020, which in turn is a continuation of application Ser. No. 16/273,850 filed Feb. 12, 2019, now U.S. Pat. No. 10,806,851 issued Oct. 20, 2020, which in turn is a continuation of application Ser. No. 15/336,930 filed Oct. 28, 2016, now U.S. Pat. No. 10,213,547 issued Feb. 26, 2019, which in turn is a continuation of application Ser. No. 14/581,461 filed Dec. 23, 2014, now U.S. Pat. No. 9,486,571 issued Nov. 8, 2016, which claims the benefit of U.S. Provisional Application No. 61/920,940 filed Dec. 26, 2013, each of which is incorporated herein in its entirety by reference.

### FIELD OF THE INVENTION

The present invention relates to wireless control of drug delivery devices and, more particularly, to a safety processor to increase the safety and reliability of wireless control of drug delivery devices with remote devices such as smartphones.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of devices for delivering such fluids is that of infusion pumps that have been developed for the administration of insulin and/or other medicaments for those suffering from both type I and type II diabetes. Some pumps configured as portable infusion devices can provide continuous subcutaneous insulin injection and/or infusion therapy for the treatment of diabetes. Such therapy may include the regular and/or continuous injection or infusion of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. patent application Ser. No. 13/557,163, U.S. patent application Ser. No. 12/714,299, U.S. patent application Ser. No. 12/538,018, U.S. patent application Ser. No. 13/838,617, U.S. patent application Ser. No. 13/827,707 and U.S. Patent Ser. No. 8,287,495, each of which is incorporated herein by reference in its entirety.

Such infusion pumps are often discretely located on or around a patient, such as beneath clothing or in a carrying pouch. Some infusion pumps are therefore adapted to be programmed with remote control devices that enable programming without directly interacting with a user interface of the pump. These remote controllers therefore enable a pump to be programmed more privately and comfortably. With the proliferation of handheld consumer electronic devices, such as smartphones, there is a desire to be able to utilize such devices as the remote controller for remotely programming an infusion pump device. However, medical devices and consumer electronics have vastly different safety and reliability profiles, such that use of such consumer electronic devices to program medical devices such as infusion pumps could present safety issues for the patient.

### SUMMARY OF THE INVENTION

A system and method provide for enhanced reliability and safety of programming and/or operating a medical device, such as an infusion pump, with a remote control device, such as a mobile phone (e.g., a smartphone). A safety processor acts as an intermediary device between the smartphone and the medical device to review transmissions from the smartphone prior to the transmissions being delivered to the medical device. The safety processor can determine whether the smartphone is compatible with the medical device by checking the type and version of the smartphone as well as the versions of the operating software and/or firmware resident on the phone. The safety processor can also check whether the operating command entered into the smartphone is within acceptable parameters, such as whether a bolus request for an infusion pump is within an acceptable range. If the safety processor determines, for instance, that the smartphone is approved for use with the medical device and the operating command is acceptable, the command is delivered from the smartphone to the medical device.

In one embodiment, a safety processor facilitates safe and reliable communications between a remote control device such as a smartphone and a medical device such as an infusion pump. The safety processor can include a transmitter/receiver that facilitates communications with each device and a memory that stores information regarding approved remote control devices for use with the medical device and information pertaining to acceptable parameters for use of the medical device on a patient. The safety processor receives communications from the smartphone intended as operating commands for the medical device. The safety processor utilizes the information stored in memory to determine if it is appropriate to transmit the communication on to the medical device. If the communication is acceptable, it is sent to the medical device for execution on the device. If the communication is not acceptable due to, for example, an incompatible device sending the communication or an operating command outside of acceptable parameters, an error message can be sent back to the smartphone.

In one embodiment, a programming operation for a medical device, such as an infusion pump, with a remote control device, such as a smartphone, utilizes a safety processor to ensure safe and reliable programming. A programming operation for the medical device can be performed on the smartphone and transmitted to the safety processor as a request for an operation on the medical device. The safety processor reviews the request and determines if it is acceptable by, for example, checking compatibility of phone hardware with the medical device, checking compatibility of phone software with the medical device and determining if the programmed operation is within a range of acceptable parameters for operations on the medical device. If the request is deemed acceptable, it is communicated to the medical device, executed on the medical device, and a status update can be sent to the smartphone. If the request is not acceptable, a communication indicating that the request was not executed can be sent to the smartphone.

Certain embodiments are described further in the following description, examples, claims, and drawings. These embodiments will become more apparent from the following detailed description when taken in conjunction with the accompanying exemplary drawings.

### BRIEF DESCRIPTION OF THE DRAWINGS

FIG. 1 is a medical device that can be utilized with embodiments of the present invention.

FIG. 2 is a block diagram representing a medical device that can be used with embodiments of the present invention.

FIG. 3 depicts an exemplary screen shot of a home screen page of a user interface of a medical device such as an infusion pump that can be used with embodiments of the present invention.

FIG. 4 is schematic representation of a system for remotely controlling a medical device according to an embodiment of the present invention.

FIG. 5 is a block diagram representing a safety processor according to an embodiment of the present invention.

FIG. 6 is a flowchart of a method of facilitating safe and reliable communications between a medical device and a remote control device.

### DETAILED DESCRIPTION

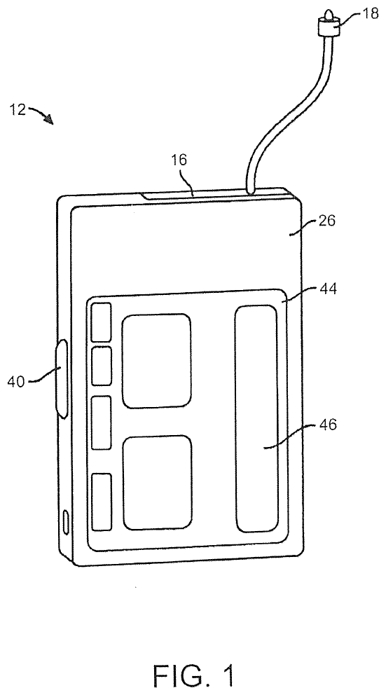

FIG. 1 depicts an embodiment of a medical device that can be used with embodiments of the present invention. In this embodiment, the medical device is configured as a pump 12 such as an infusion pump that can include a pumping or delivery mechanism and reservoir for delivering medicament to a patient and an output/display 44. The type of output/display 44 may vary as may be useful for a particular application. The type of visual output/display may include LCD displays, LED displays, plasma displays, graphene-based displays, OLED displays and the like. The output/display 44 may also be an interactive or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to a secondary display device such as a remote display, a remote control device, a laptop computer, personal computer, tablet computer, mobile communication device such as a smartphone or personal digital assistant (PDA) or the like.

In one embodiment, medical device can be a portable insulin pump configured to deliver insulin to a patient. Further details regarding such pump devices can be found in U.S. Patent Publication No. 2011/0144586, which is incorporated herein by reference in its entirety. In other embodiments, medical device can be an infusion pump configured to deliver one or more additional or other medicaments to a patient. In a further embodiment, the medical device can be a glucose meter such as a continuous glucose monitor. Further detail regarding such systems and definitions of related terms can be found in, e.g., U.S. Pat. Nos. 8,311,749, 7,711,402 and 7,497,827, each of which is hereby incorporated by reference herein in its entirety. In other embodiments, the medical device can monitor other physiological parameters of a patient.

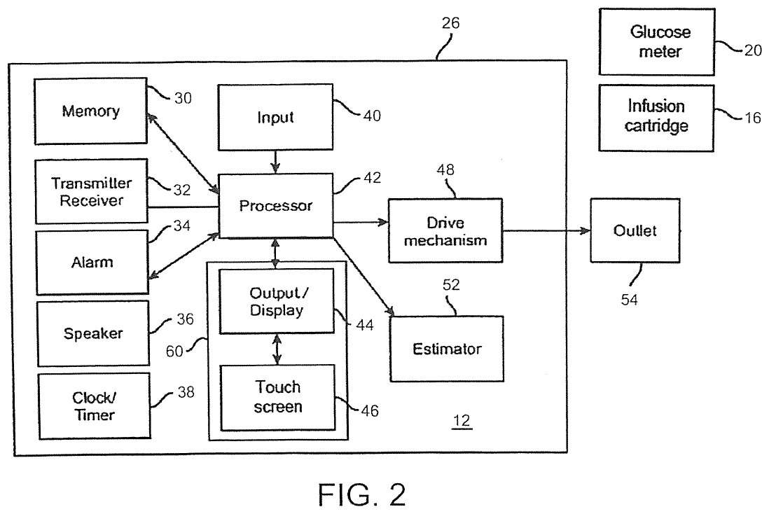

FIG. 2 illustrates a block diagram of some of the features that can be used with embodiments of the present invention, including features that may be incorporated within the housing 26 of a medical device such as a pump 12. The pump 12 can include a processor 42 that controls the overall functions of the device. The infusion pump 12 may also include a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from an input device, such as a sensor that may sense pressure, temperature or other parameters. In some embodiments, the processor 42 can communicate with a safety processor as disclosed herein.

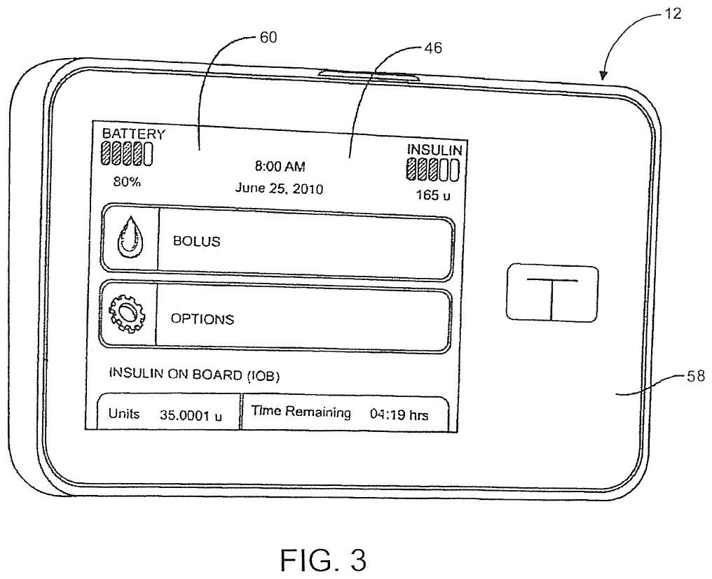

Referring to FIG. 3, a front view of pump 12 is depicted. Pump 12 may include a user interface, such as, for example, a GUI 60 on a front surface 58 or other location of pump 12. GUI 60 may include a touch-sensitive screen 46 that may be configured for displaying data, facilitating data and/or command entry, providing visual tutorials, as well as other interface features that may be useful to a caregiver or to the patient operating pump 12. The GUI can also present alarms or alerts to the user.

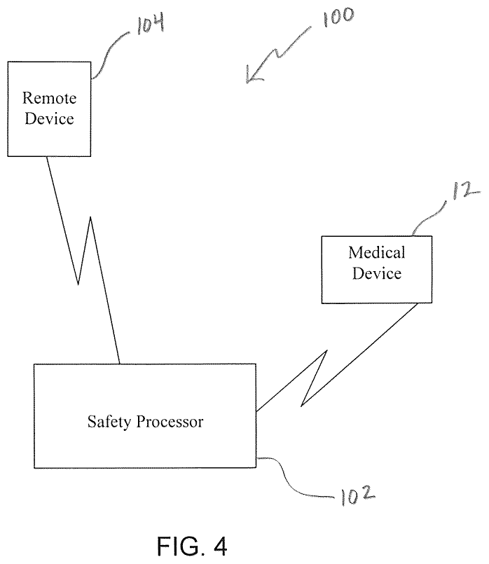

Referring now to FIG. 4, a schematic representation of a system for facilitating safe and reliable communications between a medical device and an electronic device, such as a consumer electronic device, is depicted. System 100 includes a safety processor 102 that acts as an intermediary device between the medical device 12 and a consumer electronic device functioning as a remote control device 104 for the medical device 12. Operating and/or programming commands sent from the remote control device 104 and intended for execution by the medical device are first received by the safety processor 102 where they are reviewed before potentially being sent on to the medical device 12. In one embodiment, communications between the remote device 104 and the safety processor 102 and between the medical device 12 and the safety processor 102 are done wirelessly via, for example, Bluetooth®, Bluetooth® low energy, mobile or Wi-Fi communications. Alternatively or additionally, one or both of the medical device 12 and remote device 104 can communicate with the safety processor 102 over a wired connection. In certain embodiments, the remote device 104 and medical device 12 are precluded from communicating directly with each other such that all or selected communications must first go through the safety processor 102 before being relayed to the other device. In one embodiment, remote device 104 is a mobile phone such as a smartphone. In other embodiments, remote device 104 can be any device capable of running an application or other software adapted to enable programming of operating parameters for medical device 12 and communications with safety processor 102, such as, for example, an electronic tablet or a laptop or desktop computer.

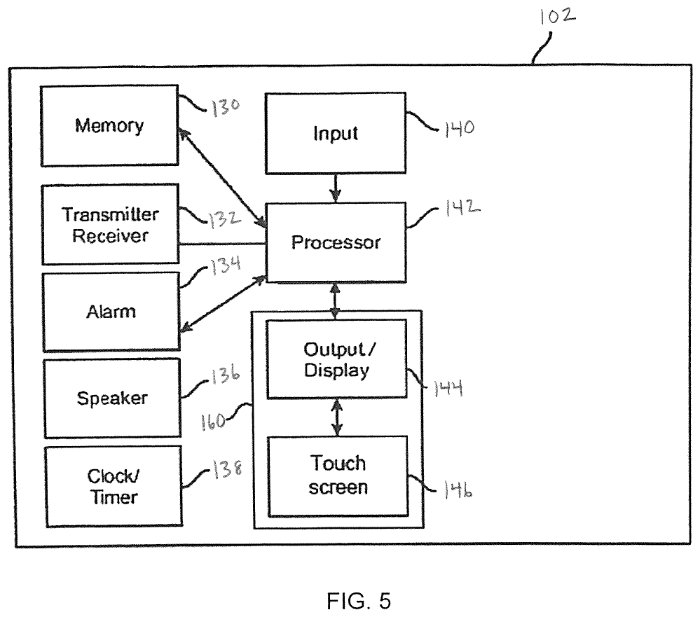

FIG. 5 depicts a block diagram of some features and components that can be associated with the safety processor 102. Safety processor 102 can include a processor 142 that controls the overall functions of the safety processor 102. The safety processor 102 can also include a memory device 130, a transmitter/receiver 132, an alarm system 134, a speaker 136, a clock/timer 138 an input device 140, and a user interface 160 having a display 144 and that, in some embodiments, can include a touch sensitive screen 146 with input capability. The processor 142 can communicate with the processor 42 of the pump 12 and a processor of the remote device 104 In some embodiments, the processor 142 may communicate with one or more processors of additional devices, for example, a continuous glucose monitor (CGM), display device, etc. through the transmitter/receiver. The processor 142 may also include programming that may allow the processor to receive signals and/or other data from an input device, such as a sensor that may sense pressure, temperature or other parameters.

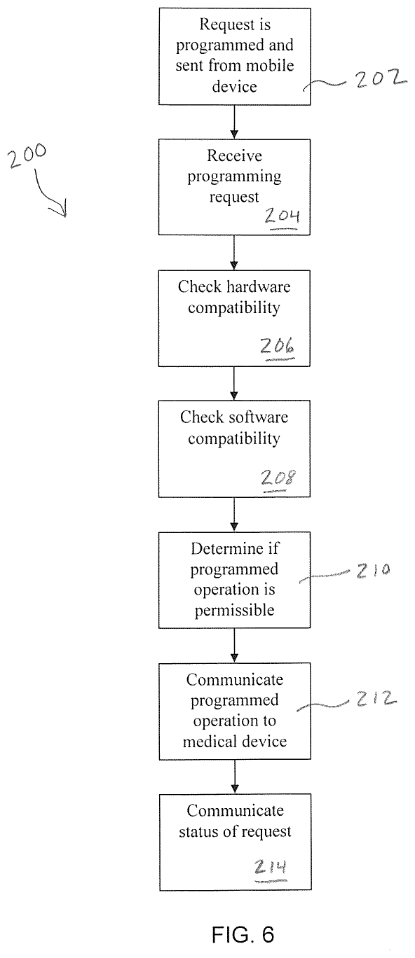

Referring now to FIG. 6, a flowchart is depicted of one example of a communications sequence 200 for operating medical device 12, such as an insulin pump, with a remote control device 104 through safety processor 102. At step 202, an operation request is entered into the remote control device 104 and transmitted to the safety processor 102. The operation request can relate to any type of operation that could otherwise be entered using the pump user interface or a dedicated remote controller such as, for example, delivery of a bolus, delivery or modification of a basal rate, review of pump history, programming of alarms, etc. The safety processor 102 receives the operation request at step 204. At steps 206 and 208, the safety processor 102 reviews the compatibility of the remote device 104 requesting the operation based on parameters stored in the safety processor memory 130. This can include hardware, software and/or firmware compatibility, such as whether the type of device is a device approved for use for controlling the medical device 12 and whether the type and/or version of the software being operated on the device for making the requests is approved.

Upon confirming compatibility of the remote device hardware, software and/or firmware, at step 210 the safety processor 102 can review the specific operation request in view of acceptable operation parameters or guidelines stored in memory to determine if the operation is permissible. For example, if the operation request is for delivery of a bolus of insulin or other medicament, the safety processor 102 can determine, for example, whether the size of the bolus is permissible, e.g., is below a maximum bolus value, and is being requested at an acceptable time after a previous bolus request. Similarly, a request to begin or modify basal insulin or other medicament delivery can be reviewed to determine if it is within an acceptable range. In certain embodiments, the acceptable parameters stored in the safety processor memory can be patient specific parameters. If the operation is permissible, safety processor 102 then communicates the programmed operation to the medical device 12 at step 212 and the medical device initiates the operation. The safety processor 102 can also monitor the operation to confirm its progress and determine when it is completed. In some embodiments, the safety processor 102 can require a confirmation from the remote device 104 prior to communicating the operation to the medical device 12. Finally, at step 214 the safety processor 102 can communicate with the remote device 104 to confirm the status of the request, including that the operation is in progress and/or completed, or that the request failed. In some embodiments, if a request has failed the safety processor 102 can provide a reason for the failure to the remote device 104, such as, for example, the bolus requested was larger than a maximum permissible bolus or the remote device 104 is not approved due to hardware, software and/or firmware incompatibility, etc.

In certain embodiments, safety processor 102 can also provide a number of other functions in addition to reviewing and transmitting communications between mobile device 104 and medical device 12. For example, as noted previously safety processor 102 can include an alarm system 134, speakers 136 and user interface 160. These features can be employed as part of a patient notification system. Such a system can include one or more of vibratory, audio (including natural language) and visual indicators to the patient of the progress of requests and/or errors, providing redundant and/or alternative notifications to those that may appear on the mobile device 104.

Safety processor 102 may be able to update its own software to, for example, provide an updated database of acceptable mobile device hardware and software/firmware versions with which the medical device is compatible and/or update the permissible operating parameters for the medical device. Such an update could be accomplished remotely through a wireless connection or through a wired connection. In some embodiments, software/firmware updates can occur automatically. In other embodiments, the safety processor 102 may notify the user through, for example, the user interface 160 of the safety processor 102 and/or the mobile device 104, that a software and/or firmware update is available and request that the user initiate the update at the safety processor 102 or the mobile device 104. In addition, in some embodiments the mobile device 104, prior to a software and/or firmware upgrade, may be able to communicate with the safety processor 102 to have the safety processor 102 confirm that the upgrade will be compatible with the system to allow programming of the medical device 12.

In some embodiments, the safety processor 102 can include features enabling programming of the medical device 12 directly from the safety processor 102. This can provide an additional means for operation of the medical device 12 if the remote device 104 is unavailable or has been deemed or determined to be incompatible with the medical device 12. The safety processor 102 can be provided with functionality to control all aspects of the medical device 12, or with a more limited set of features. For example, in one embodiment, the safety processor is only capable of programming a bolus of medicament such as insulin for delivery to the patient.

Safety processor 12 can also communicate with alternative or additional devices and, in some embodiments, act as an intermediary between the remote device 104 and those other devices and/or the medical device 12 and those other devices. Such devices include, for example, glucose meters, continuous glucose monitors and monitors for other physiological parameters.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 8,287,495; 8,408,421 and 8,448,824; commonly owned U.S. Patent Publication Nos. 2009/0287180; 2010/0008795; 2010/0071446; 2010/0218586; 2012/0123230; 2013/0053816; 2013/0159456; 2013/0306191; 2013/0324928; 2013/0332874; 2013/0283196; 2013/0331790; 2013/0331778; 2014/0276531; 2014/0276419; 2014/0276420; 2014/0276423; 2014/0276,409; 2014/0276537; 2014/0276553; 2014/0276569; 2014/0276570; 2014/0276571; 2014/0276574; 2014/0276556; 2014/0276538; and commonly owned U.S. patents application Ser. Nos. 13/923,556; 14/479,994; and 14/482,521 and commonly owned U.S. Provisional Application Ser. Nos. 61/911,576, 61/920,902, 61/920,914, 61/920,932; 61/920,940; 61/990,501; and 62/030,933.

Further incorporated by reference herein in their entirety are U.S. Pat. Nos. 8,601,465; 8,502,662; 8,452,953; 8,451,230; 8,449,523; 8,444,595; 8,343,092; 8,285,328; 8,126,728; 8,117,481; 8,095,123; 7,999,674; 7,819,843; 7,782,192; 7,109,878; 6,997,920; 6,979,326; 6,936,029; 6,872,200; 6,813,519; 6,641,533; 6,554,798; 6,551,276; 6,295,506; and 5,665,065.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A method of enabling control of an ambulatory infusion pump, comprising:
determining whether software operating on a remote electronic device is compatible with a software application configured to enable the remote electronic device to communicate operating commands to the ambulatory infusion pump;
enabling the remote electronic device to communicate operating commands for control of the ambulatory infusion pump if the software is compatible; and
causing the remote electronic device to display an error message if the software is not compatible.

2. The method of claim 1, wherein determining whether the software operating on the remote electronic device is compatible includes determining whether a type of the software is compatible.

3. The method of claim 1, wherein determining whether the software operating on the remote electronic device is compatible includes determining whether a version of a type of software is compatible.

4. The method of claim 1, wherein causing the remote electronic device to display the error message includes causing the error message to inform the user that the software is not compatible.

5. The method of claim 1, further comprising maintaining a database of compatible software.

6. The method of claim 5, further comprising updating the database of compatible software to include additional software.

7. The method of claim 1, further comprising determining whether or not the remote electronic device is compatible for use to communicate operating commands to the ambulatory infusion pump.

8. The method of claim 7, wherein determining whether or not the remote electronic device is compatible includes determining compatibility of a type of the remote electronic device.

9. The method of claim 8, wherein determining compatibility of the type of the remote electronic device includes determining compatibility of a version of the type of the remote electronic device.

10. The method of claim 1, further comprising determining whether a type of firmware operating on the remote electronic device is compatible for use to communicate operating commands to the ambulatory infusion pump.

11. A method of enabling control of an ambulatory infusion pump, comprising:
downloading a software application onto a remote electronic device, the software application configured to enable the remote electronic device to communicate operating commands to an ambulatory infusion pump;
receiving input into the software application for programming an operating command for the ambulatory infusion pump;
determining whether a software operating on the remote electronic device is compatible with the software application; and
displaying an error message on the remote electronic device in response to the input if the software is not compatible.

12. The method of claim 11, wherein determining whether the software operating on the remote electronic device is compatible includes determining whether a type of the software is compatible.

13. The method of claim 11, wherein determining whether the software operating on the remote electronic device is compatible includes determining whether a version of a type of software is compatible.

14. The method of claim 11, wherein displaying the error message includes informing the user that the software is not compatible.

15. The method of claim 11, further comprising maintaining a database of compatible software.

16. The method of claim 15, further comprising updating the database of compatible software to include additional software.

17. The method of claim 11, further comprising determining whether or not the remote electronic device is compatible for use to communicate operating commands to the ambulatory infusion pump.

18. The method of claim 17, wherein determining whether or not the remote electronic device is compatible includes determining whether a type of the remote electronic device is compatible.

19. The method of claim 18, wherein determining compatibility of the type of the remote electronic device includes determining compatibility of a version of the type of the remote electronic device.

20. The method of claim 11, further comprising determining whether a type of firmware operating on the remote electronic device is compatible for use to communicate operating commands to the ambulatory infusion pump.
